(19) United States
(12) Patent Application Publication
(10) Pub. No.: US 2012/0165703 A1
(43) Pub. Date: Jun. 28, 2012

(54) Preempt Muscle Map Screen

(75) Inventors: Paul William Bottum, Stillwater, MN (US); Michael Christopher Bottum, East Troy, WI (US); Aaron Leventhal, Minneapolis, MN (US)

(73) Assignee: Paul William Bottum, Stillwater, MN (US)

(21) Appl. No.: 13/329,323

(22) Filed: Dec. 18, 2011

Related U.S. Application Data
(60) Provisional application No. 61/425,760, filed on Dec. 22, 2010.

(51) Int. Cl. A61B 5/11

(52) U.S. Cl. 600/595

(57) ABSTRACT

Some embodiments of the invention provide a software generated series of tests or screens to determine skeletal muscle balance as related to muscle strength and muscle flexibility. In some embodiments, a color coded muscle map illustrating the findings of the screens, is generated. The findings of the screens compile an exercise program, specifically tailored to the individual's general health and fitness state, as well as to the specific shortcomings identified in the screen. The invention also provides, in some embodiments, an exercise program adherence tracking calendar which monitors both completed exercises and exercise effectiveness as measured by changes in screen results.

### REFERENCE TO RELATED APPLICATIONS

This application claims priority to United States Provisional Application filed on Dec. 12, 2010, assigned Application No. 61/425,760 and titled “Preempt Muscle Map Screen”.

### REFERENCES CITED

| | | |
|---|---|---|
| 5,667,459 | Sep. 16, 1997 | Su; Li-ping |
| 8,027,822 | Sep. 27, 2011 | Turgiss, et al |
| 7,809,153 | Oct. 5, 2010 | Bravomalo, et al. |
| 6,098,458 | Aug. 8, 2000 | French; Barry James |
| 6,010,452 | Jan. 4, 2000 | Harcourt; Kristian L. |
| 4,463,946 | Aug. 7, 1984 | Wallace; Lynn A. |
| 8,075,451 | Dec. 13, 2011 | Dugan; Brian M. |
| 7,722,504 | May 25, 2010 | Younger; J. Kevin |

### OTHER REFERENCESD

(1) Yusef, S., Hawken, S., et al., 2004. Effect of potentially modifiable risk factors associated with myocardial infarction in 52 countries (the INTERHEART study): case-control study. Lancet 364.
(2) Epel, E., Lin, J., et al., 2005. Cell aging in relation to stress arousal and cardiovascular disease risk factors. Psychoneuroendocrinology 31, 277-287.
(3) Castro, C., 2002. Telephone-assisted counseling for physical activity. Exercise and Sport Sciences Reviews, 30(2), 64-68.
(4) ASCM http://www.acsm.org/about-acsm/media-room/news-releases/2011/08/01/acsm-issues-new-recommendations-on-quantity-and-quality-of-exercise.

### FIELD OF THE INVENTION

The invention is directed towards measuring, representing, and improving the musculoskeletal state of health as determined by strength, flexibility, coordination, and balance.

### BACKGROUND OF THE INVENTION

The human body, must move to maintain a healthy physical and psychological state. Studies have linked movement and general physical fitness to cardiovascular health (Yusuf, 2004), as well as biological age (Epel, 2006). As the body ages, however, obstacles arise which may prevent either the ability or motivation to maintain physical fitness. Injuries may produce chronic pain or joint immobility. Overuse or repetitive use behaviors may cause asymmetries in strength, flexibility, or classic syndromes of symptoms such as carpal tunnel or tennis elbow—any of which may inhibit motility required for the maintenance of physical fitness. Programs designed to restore compromised motility or conditions preventing it tend experience poor success rates for the following reasons;

1—The conventional Western health paradigm involves reactivity to diseased or injured states. Once symptomatic, a pathology is often widespread beyond the tissue or system of obvious injury. It is quite common that many disorders are rooted in years of underling misuse or disease.

2—The communication pathway between doctor, physical therapist, and trainer tends to be disjointed if existent at all. Due to the specialized nature with which Western medicine is practiced, a holistic approach to an individual's health is often overlooked. For example, the effect of a poorly healed broken bone on the progression of osteoarthritis, in turn on the lack of motility, in turn on the reduced ability for maintaining physical fitness, in turn on increased potential for cardiovascular disease during the lifetime of an individual may be ignored or overlooked.

3—Adherence to prescribed exercise or physical rehabilitation programs tends to be very low. Health care programs typically fund physician prescribed physical therapy for four to six weeks. Once completed, costs typically dictate that physical therapy ends. Continued fitness adherence is up to the individual. Enrollment in community or canned fitness programs tends to be poorly tailored to the individual's needs, personal conditions or individualized feedback. Studies have shown that individualized focus on fitness states and programs tends to produce the highest fitness program adherence rates (Castro, 2002). Use of a personalized physical trainers yields effective, though expensive, individualized fitness protocols, however, adherence motivation is often lacking due to the absence of tangible measures of protocol effectiveness and fitness improvement.

Several attempts have been developed for automating the quantitative determination of an individual's fitness (Wallace, 1984, French, 2000), methods to design targeted fitness programs for elite athletes (Younger, 2010), or the computation of biological age based fitness scores (Harcourt, 2000). Each of these present shortcomings to the average individual; either requiring elaborate equipment, exhaustive testing resources, lack of integration with a tailored exercise program, or lack of personalized exercise program effectiveness feedback.

There exists an apparent need for a fitness program to recognize musculoskeletal imbalances and deficits, to design and prescribe an exercise protocol tailored to the identified imbalances and shortcomings, to promote and monitor protocol adherence and finally, to provide feedback on program effectiveness and fitness improvement.

### BRIEF SUMMARY OF THE INVENTION

The Preempt Muscle Map Screen is a software based tool that provides the user with a means to easily and accurately measure deficiencies in musculoskeletal fitness, design an exercise program targeting those deficiencies, and to monitor program adherence and effectiveness as determined by improvements in fitness measures.

Identification of musculoskeletal fitness deficiencies such as muscle weakness, joint laxity, or reduced joint range of motion, is accomplished through the user comparing their ability to assume a series of fourteen body poses or screens. Each screen targets the health of specific muscle groups and joints. The user compares their own pose to idealized healthy, moderately, or severely deviating pose images. Comparisons may be made either through the use of a mirror, video input such as a webcam, camera or video game motion monitoring device, or through feedback from a partner or personal trainer.

As screens are completed, the tool compiles results and generates both a fitness score based on user anthropometric data, lifestyle and screen results, and a colorized Muscle Map, providing quantitative and qualitative representations of musculoskeletal fitness. The tool then uses the screen results to select exercises from an exercise database, specifically targeting identified deficiencies. Selected exercises are combined with instructions and dosages based on the user's fitness score and personal fitness goals, into an interactive exercise calendar.

The interactive calendar logs and monitors program adherence as exercises are completed on a day to day basis. Finally, the tool monitors fitness program effectiveness by reminding the user to re-screen and compare themselves to previous or initial Muscle Maps.

### BRIEF DESCRIPTION OF THE DRAWINGS

FIG. 1 is an example of the personal anthropometric data entered by the user.

FIG. 2 is an example of first posture of the screen determining health of right and left windows (transverse abdominals).

FIG. 3 is an example of the second posture of the screen determining flexibility of core/transverse abdominals.

FIG. 4 is an example of the third posture of the screen measuring flexibility of shoulder muscle group (deltoid, trapezius, latissimus dorsi).

FIG. 5 is an example of the fourth posture of the screen measuring health of transverse abdominals, hip flexors, gluteus maximus, and hamstrings.

FIG. 6 is an example of the fifth posture of the screen, measuring health of core muscles (transverse and rectus abdominals, latissimus dorsi, gluteus maximus and hamstrings.

FIG. 7 is an example of the sixth posture of the screen, measuring flexibility of hamstrings and gluteus maximus and strength of abdominals and hip flexors

FIG. 8 is an example of the seventh posture of the screen, measuring health of gluteus maximus, thigh and rectus abdominals.

FIG. 9 is an example of the eighth posture of the screen, measuring health of rectus abdominus, hip flexors, hamstrings and gluteus maximus.

FIG. 10 is an example of the ninth posture of the screen, measuring health of quadriceps and gastrocnemius.

FIG. 11 is an example of the tenth posture of the screen, measuring health of rectus abdominus, quadriceps, and the outer thigh muscle group.

FIG. 12 is an example of the eleventh posture of the screen, measuring health of the rectus abdominus, outer thigh and hip muscle groups, and quadriceps.

FIG. 13 is an example of the twelfth posture of the screen, measuring health of transverse abdominals, hip flexors, quadriceps and hamstrings.

FIG. 14 is an example of the thirteenth posture of the screen, measuring strength of the latissimus dorsi.

FIG. 15 is an example of the fourteenth posture of the screen, measuring strength of core muscle groups, external/internal obliques, transverse abdominals, rectus abdominus, gluteus maximus, and hip flexor muscle groups.

FIG. 16 is an example of how the user would choose their personalized exercise program.

FIG. 17 is an example of the fitness score and muscle map that are generated based on the user's anthropometric data and results of the screen postures.

FIG. 18 is an example of the muscle map and exercise program generated for the user based on user goals and screen posture results.

FIG. 19 is an example of how muscle maps from different screening dates may be compared to indicate fitness improvement.

FIG. 20 is an example of the user's exercise calendar.

FIG. 21 is an example of the instructions and images which accompany each exercise.

FIG. 22 is an example of how the user may enter daily health state.

FIG. 23 is an example of how completions of the user's daily prescribed exercises would be logged as adherence to their exercise program.

FIG. 24 is an example of how the user could access and choose exercises from the database of all exercises to further refine their fitness program.

FIG. 25 is an example of the muscle map that male users would see when using the PREEMPT software.

### DETAILED DESCRIPTION OF THE INVENTION

It should be noted from the outset that the fitness measuring and training protocols described herein require resources available to any individual user. The body pose images and instructions contained in each fitness screen allow simple, accurate and reproducible self-administration of fitness measurement protocols. Fitness measurement results are presented in both quantitatively and qualitatively easily understandable displays, which allow for tangible fitness goal setting and improvement recognition. The exercises are designed such that, the visual, auditory and written cues provided, combined with user fitness level appropriate dosages, ensure effective targeting of deficient muscle groups or skeletal components, within a common home setting.

The following description details three main components of the invention.

They are;
1. a description of the fitness screening protocol,
2. an explanation of the generation of the Preempt Fitness Score and Muscle Map,
3. an explanation of the exercise program created based on Fitness Score and Muscle Map results.

### Fitness Screening Protocol

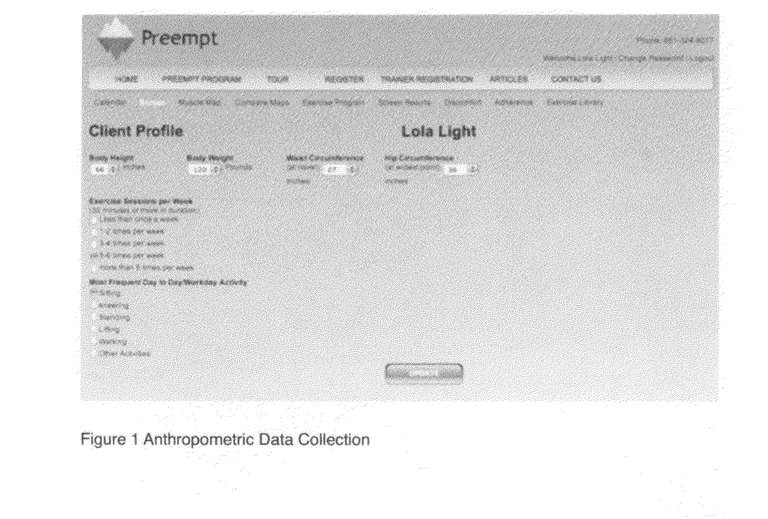

As a first time user of the Preempt tool, an individual enters anthropometric data including body height, body mass, waist and hip measurements, gender and age. Behavioral patterns such as leisure and work physical activities are noted as well (FIG. 1).

Upon completion of personal information data entry, the tool coaches the user through a series of fourteen body poses or screens. The body poses called for in each screen target specific muscles, muscle groups, coordination patterns, or skeletal components. In order for the user to accurately assume a pose, healthy levels of muscle strength, muscle coordination or balance, and joint flexibility is required. Healthy levels of muscle and joint function are based on values set by the American College of Sports Medicine (ASCM 2011).

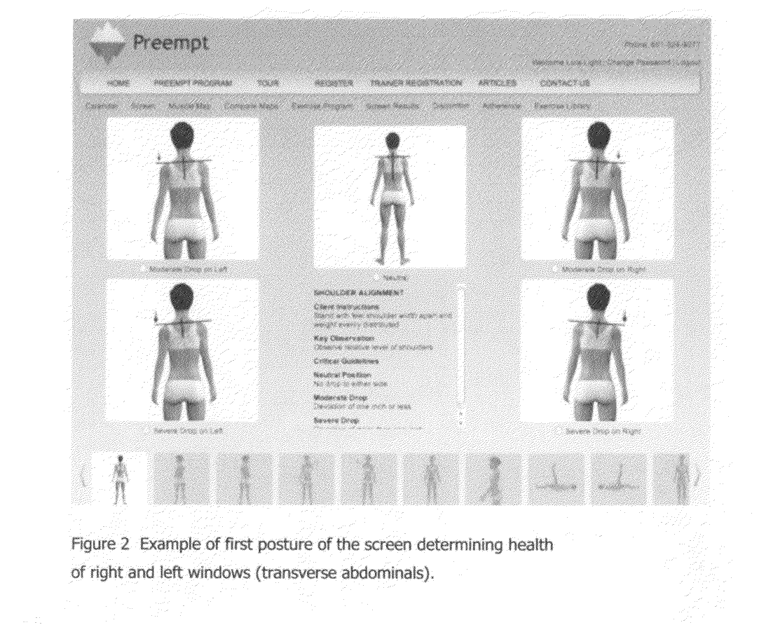

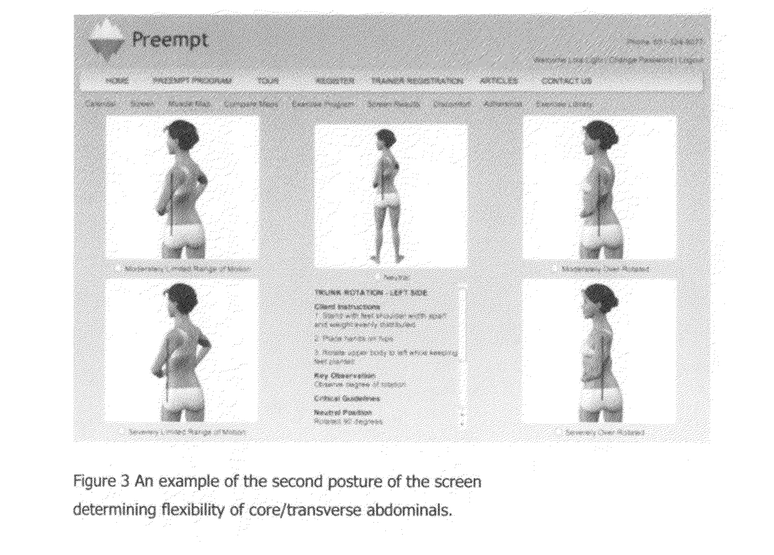

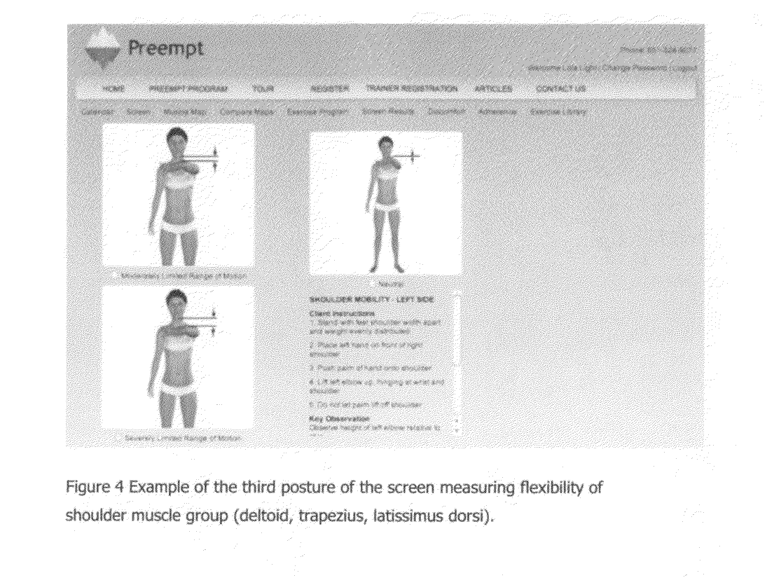

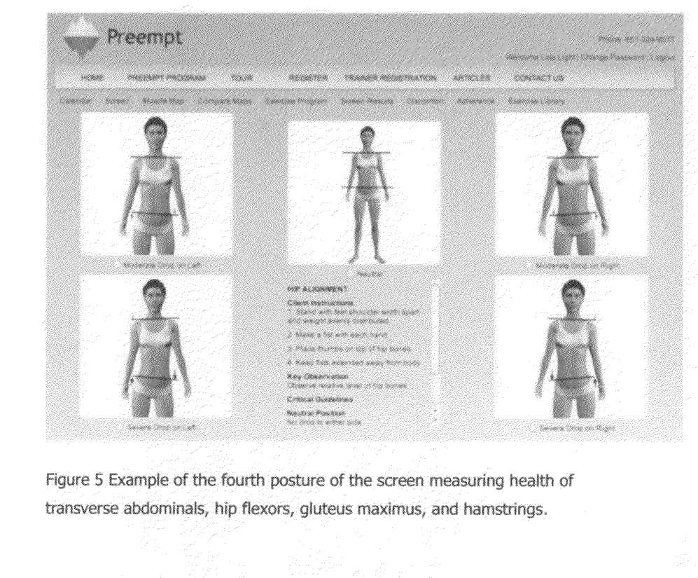

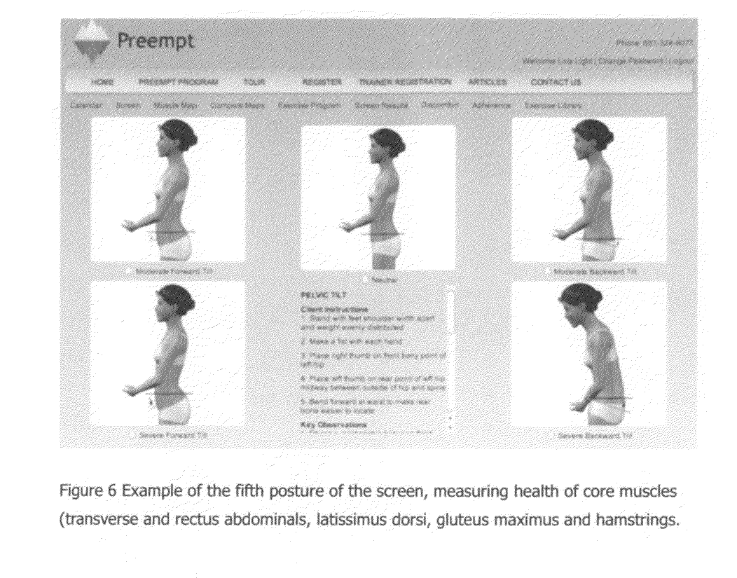

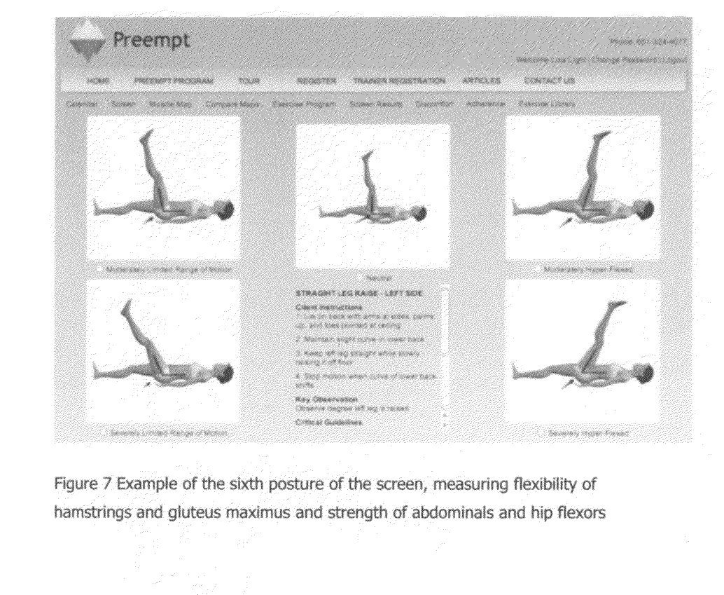

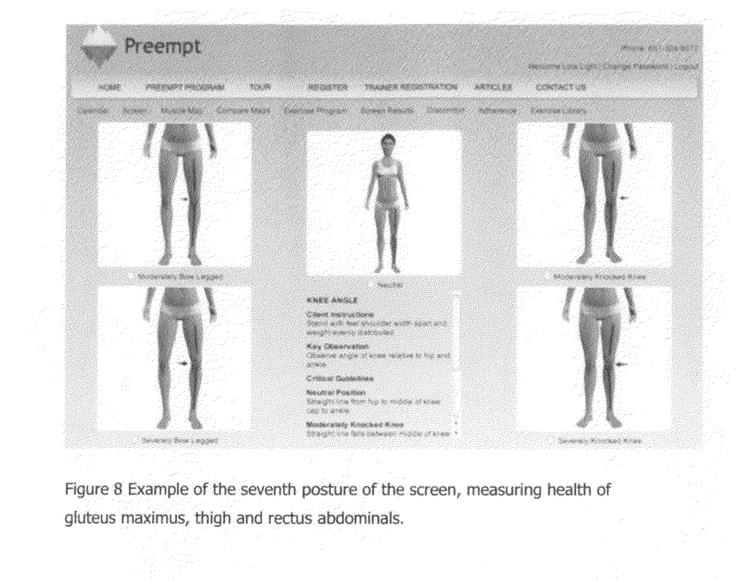

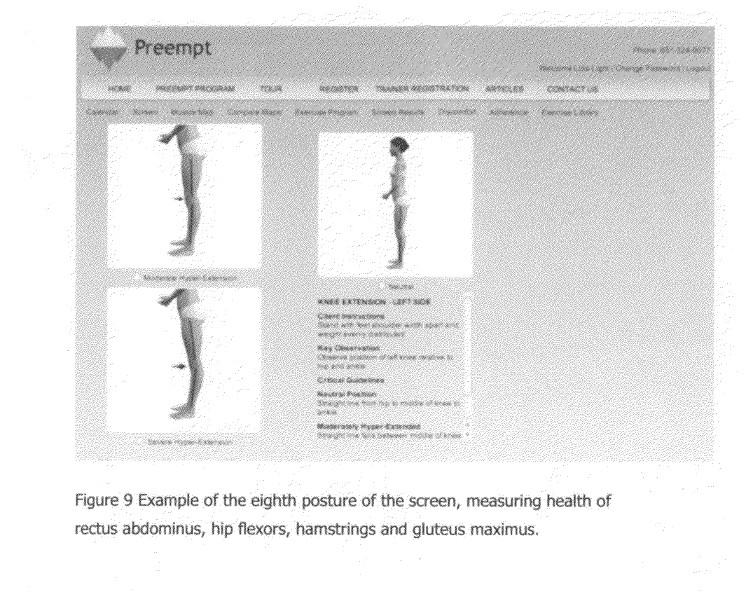

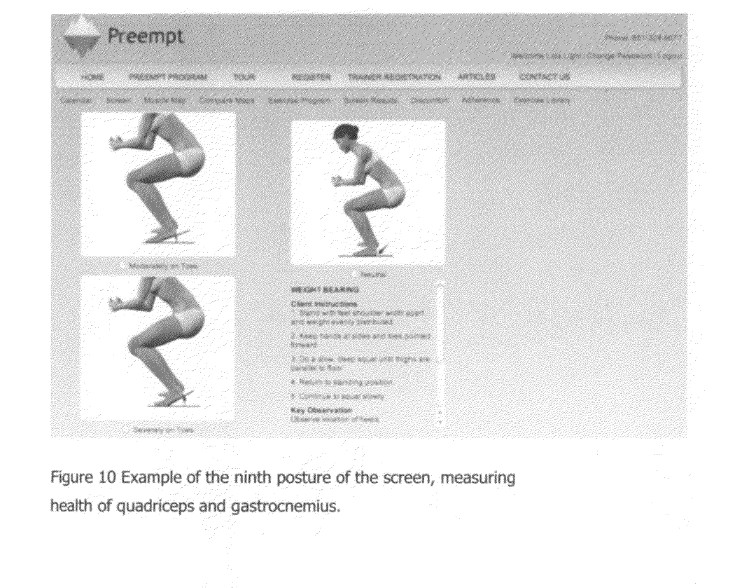

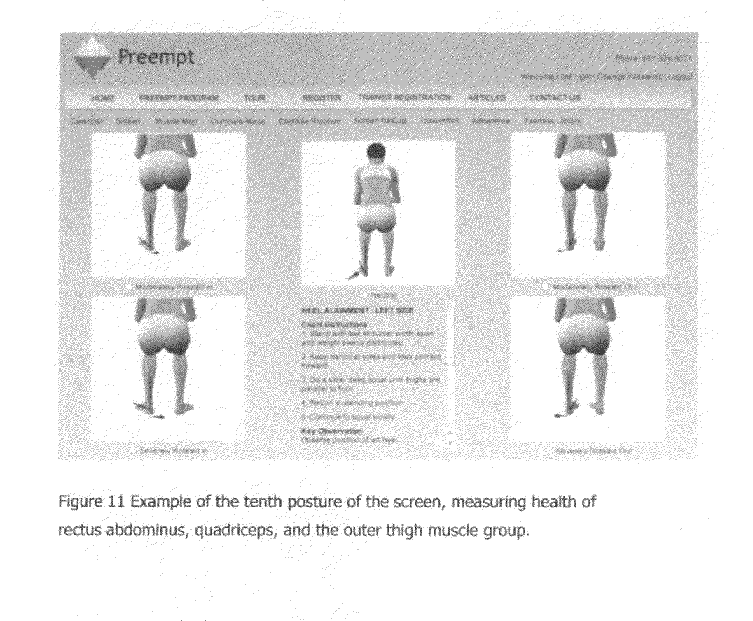

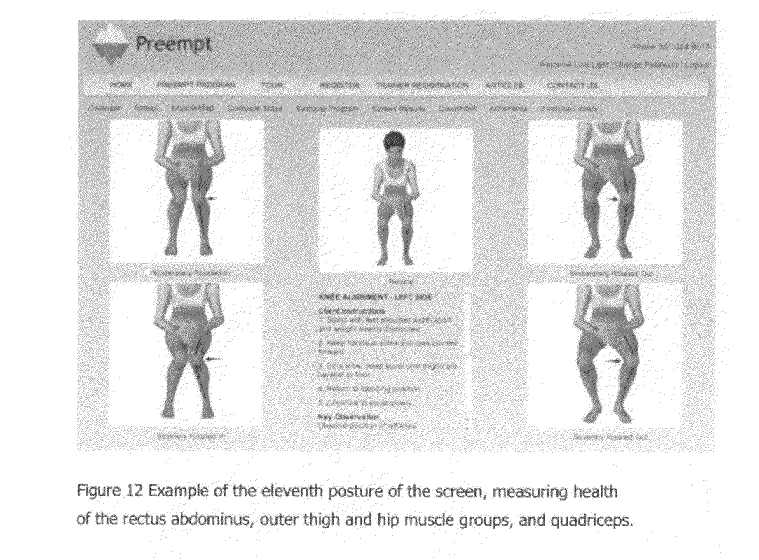

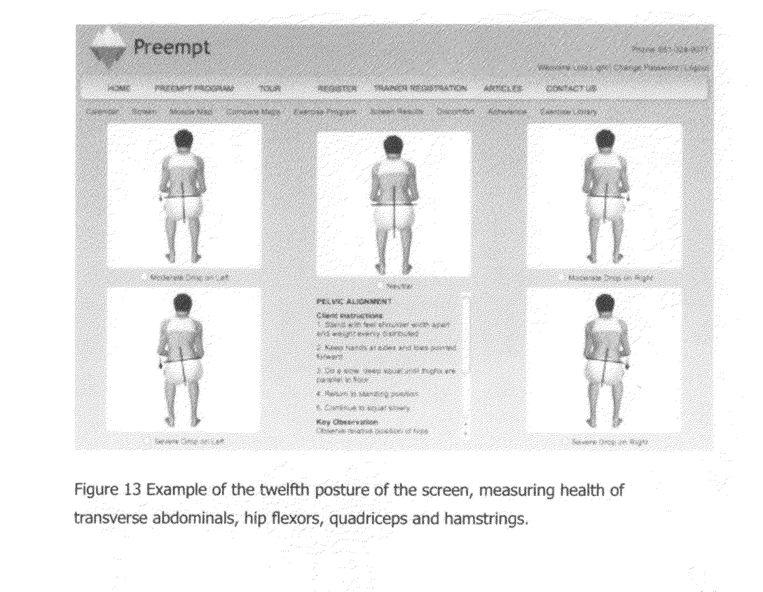

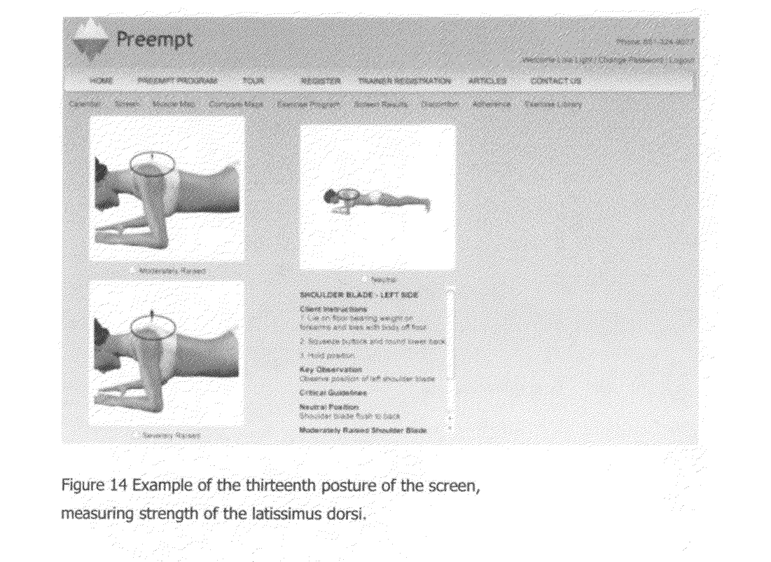

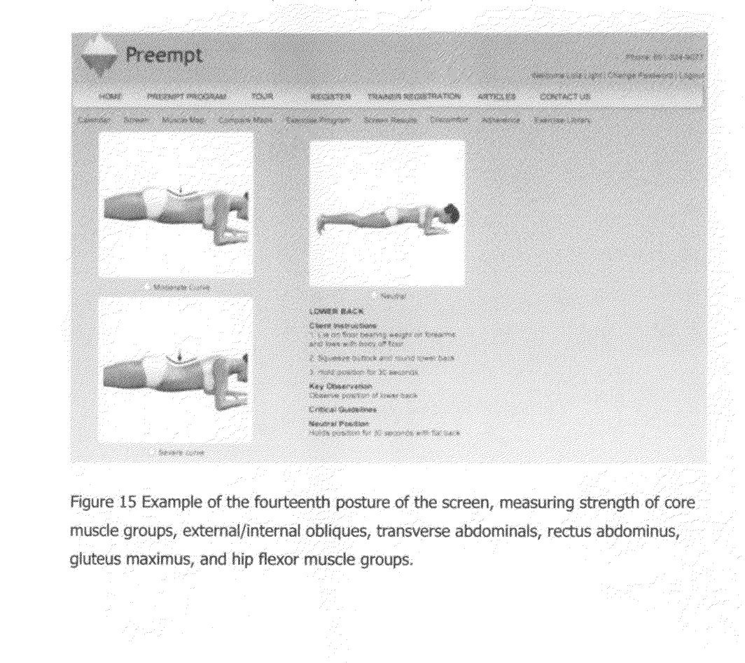

Users may monitor their ability to assume each pose through self observation in a mirror, imaging device, or personal trainer. Observations are compared to images of each screen demonstrating ideal posture, moderate deviations from ideal, or severe deviations. Images most closely representing the users posture for each screen are chosen as the user progresses through the screening protocol. FIG. 2 represents ideal and deviant poses for screen number one, which isolate the function of the transverse abdominal muscles. See FIGS. 3-15 for examples the remaining thirteen screens, containing ideal, moderate or severely deviant posture images, and descriptions of target muscles.

### Generation of the Preempt Fitness Score and Muscle Map

The Preempt Fitness Score is intended to provide a quantitative measure of general health fitness. The Fitness Score is based on an ideal health of 100. A Fitness Score of 100 represents an individual, aged twenty-five, with a healthy musculoskeletal system, and conforming to fitness promoting leisure and work related activities as defined by the ACSM (ASCM 2011). Actual Fitness Scores are normalized to a user's age, health risk factors, lifestyle and screen results based on published and accepted values (Yusef 2004, ASCM 2011). The Fitness Score serves two functions for tool. Firstly, prescribed exercise dosages are based on the Fitness Score. For example, age and lifestyle may prevent safe engagement of advanced exercises even though the screen might indicate healthy muscle groups. The Fitness Score would recognize this circumstance. Secondly, the Fitness Score provides a simple measure of fitness, through which fitness improvement may be recognized in subsequent screens.

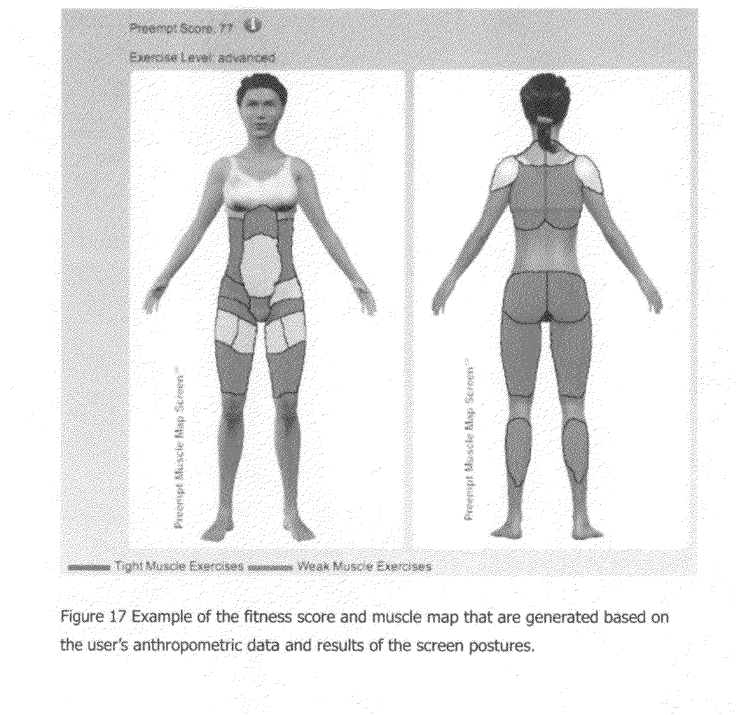

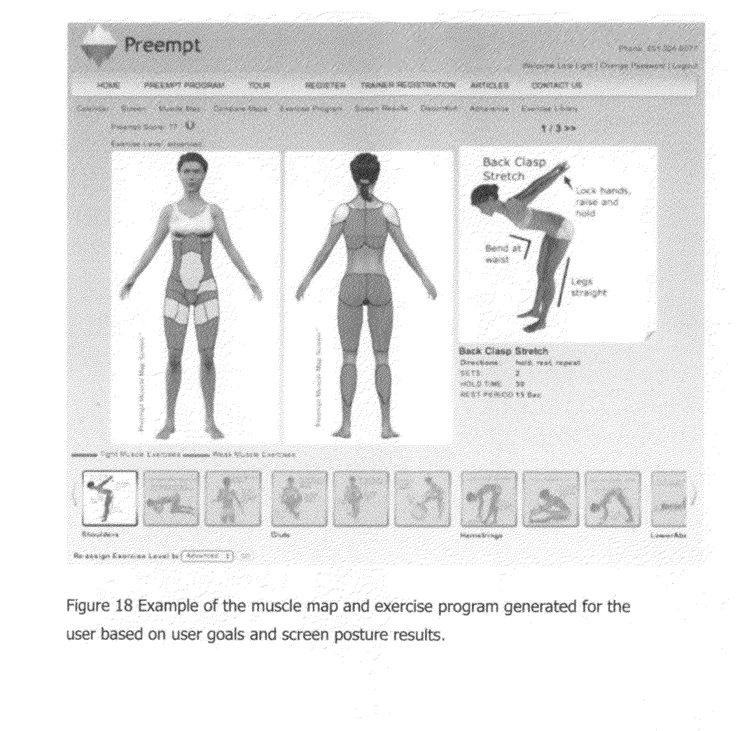

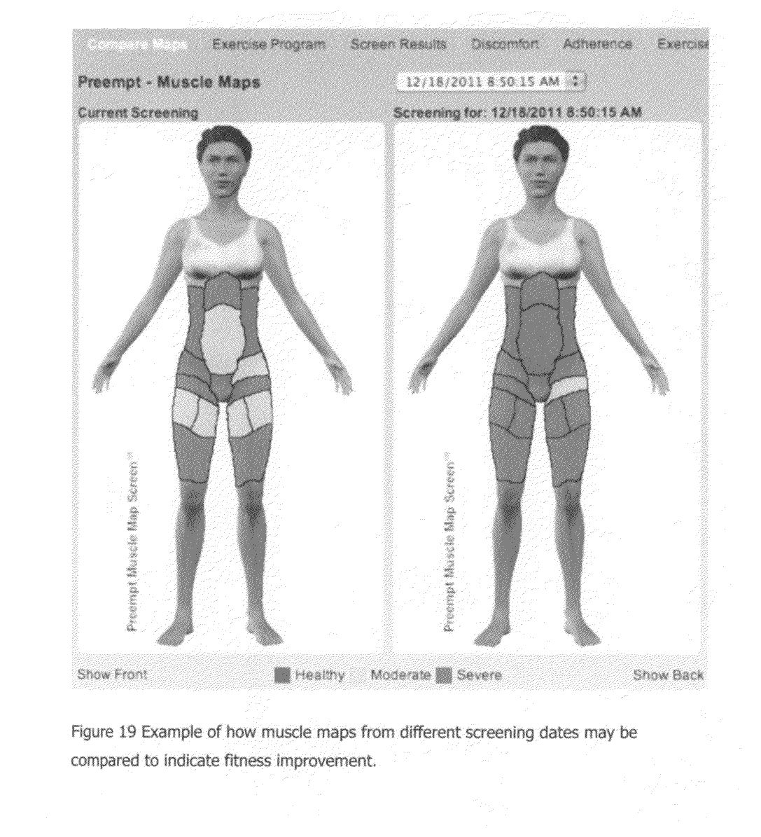

The Preempt Muscle Map provides a color coded, intuitively understandable, representation of the user's body. Based on screen results, muscles, muscle groups or skeletal components are painted green yellow or red, corresponding to healthy, moderately, or severely deficient states. FIG. 17 and FIG. 19 provide examples of how either female or male users would view representation of their screen results. It should be noted that the health state of any colored region represents the compilation of results from multiple screens. For example, the health state or color of the core windows region (rectus abdominus) is determined by combinations of results from screens one, two, four, five, twelve and fourteen. The redundancy provided through multiple screen results ensures accurate and reproducible determination of the health state of each body region.

The Muscle Map provides a visual, highly specific, qualitative representation of musculoskeletal health. Any deficient body region states or imbalances become immediately apparent. Comparison of maps from subsequent screening sessions provides the user with an accurate measure of exercise program effectiveness and improvement.

### Preempt Exercise Program.

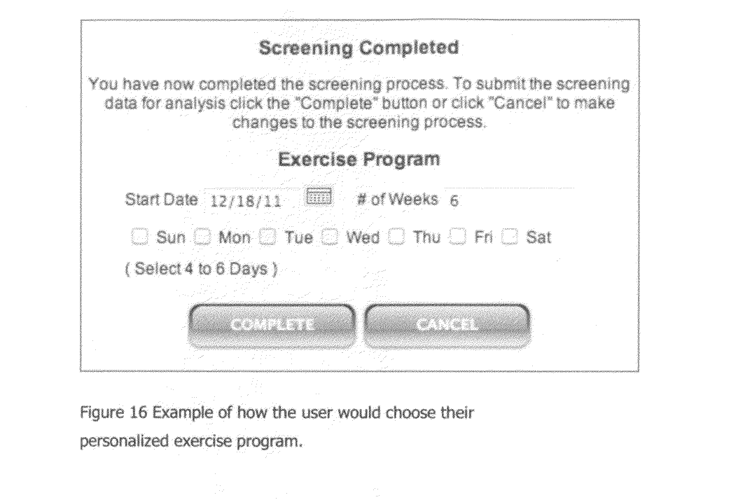

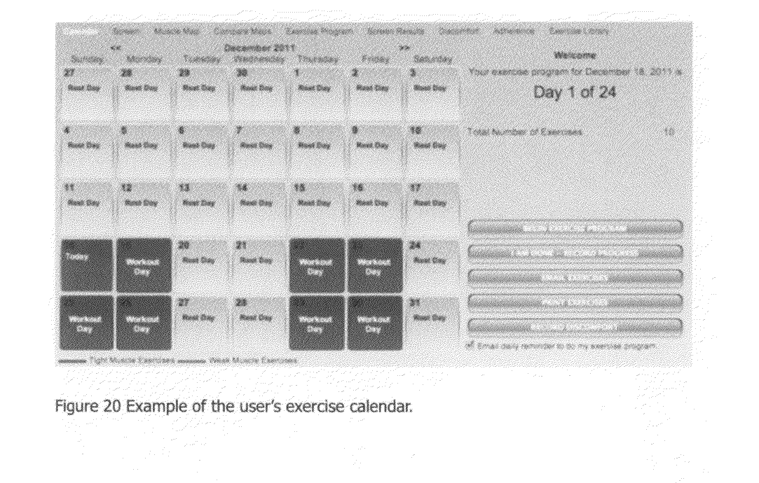

As stated in paragraphs [0025] and [0030], screen results and the Fitness Score drive the selection and dosing of body region specific exercises. Upon completion of the final screen, fourteen in this embodiment, the user choses preferred times and weekly frequencies of exercise (FIG. 16). The tool then compiles an exercise list targeting both improvement to deficient and maintenance of healthy body regions. The exercise list is integrated into an interactive exercise calendar based on user preferences (FIG. 20). The calendar is then stored either on a home computer, or uploaded to tablets or smart phone devices. Exercises are automatically shuffled on a day to day basis ensuring that the most effective physiological response (training effect) is experienced by each body region.

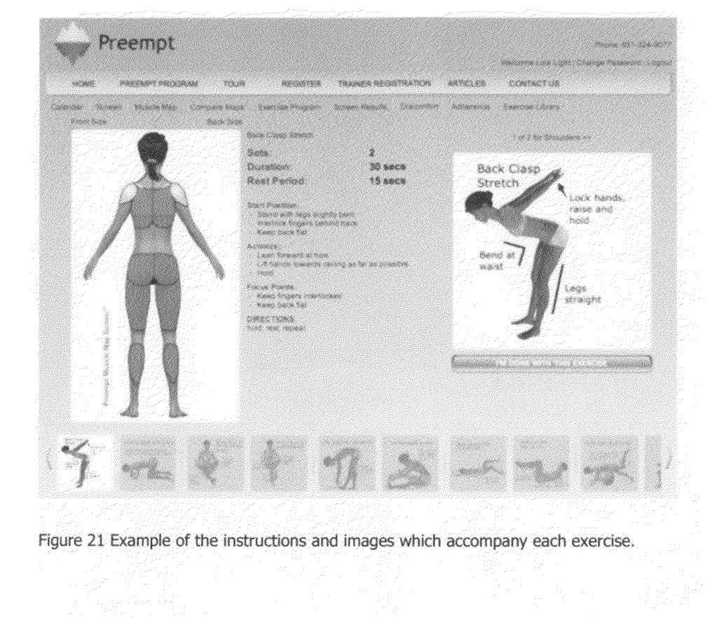

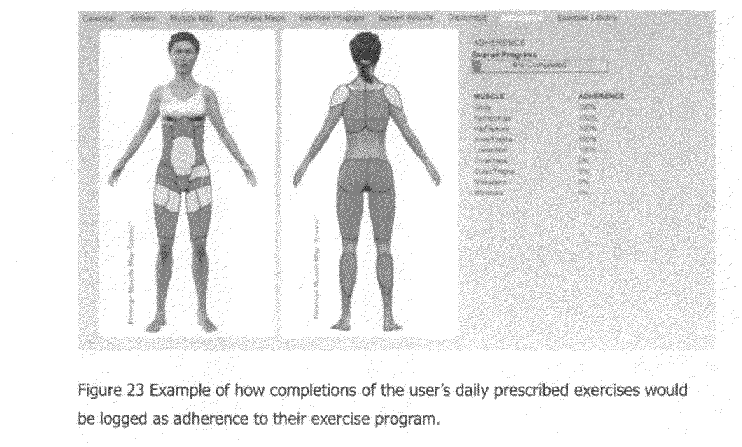

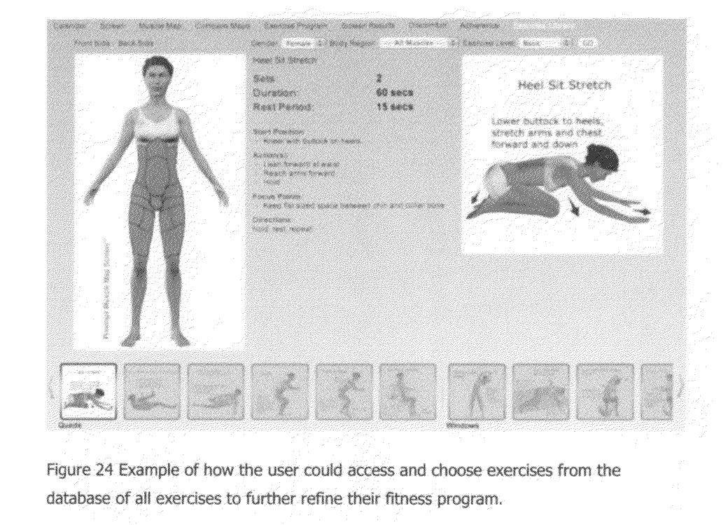

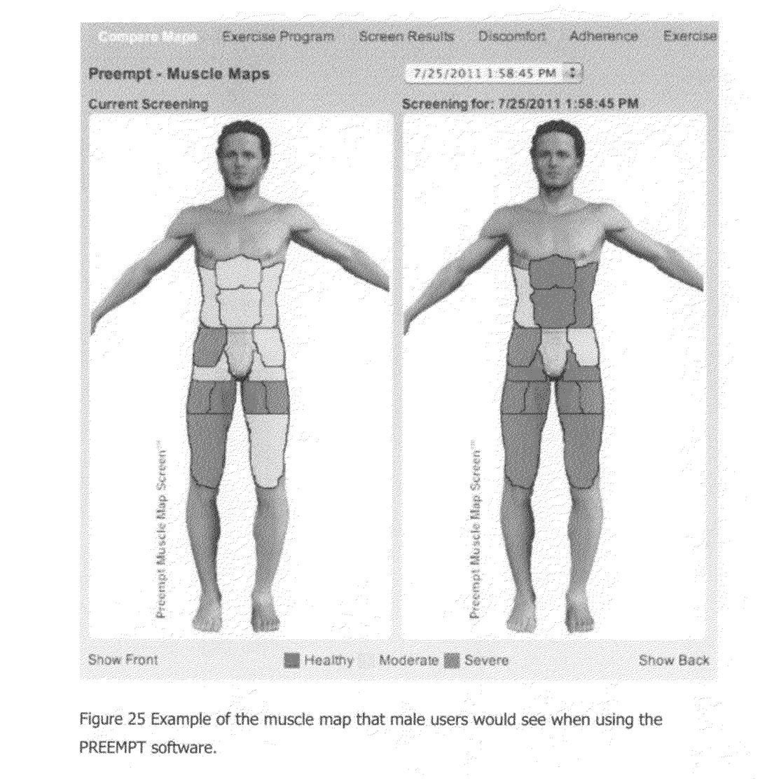

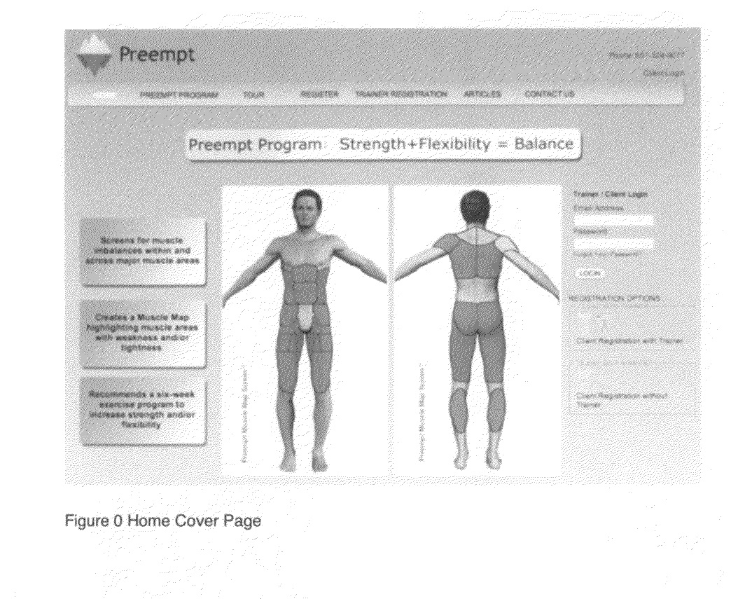

For a given exercise day, as the training session is selected on the interactive calendar, visual, auditory and written queues are provided to coach the user through each exercise (FIG. 21). User entered conformation of each exercise automatically enters it into the user's adherence log (FIG. 23).

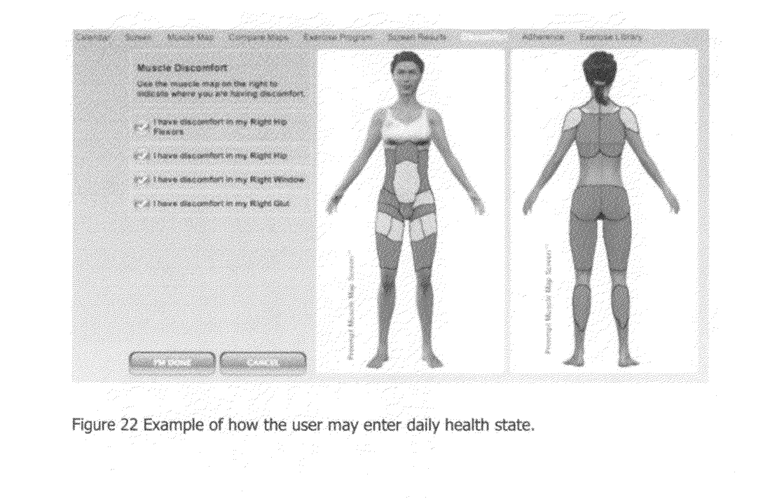

FIG. 22 provides an example of how a user might log daily health. Pain experienced in muscle groups or joints may indicated overtraining or incorrect exercise technique. Information logged in this manner may be used to refine the user's exercise program. FIG. 23 provides an example of how a user would access the exercise database to select further exercises selectable by degree of difficulty, targeted body region, or fitness goals.

Finally, using the Preempt Fitness Score and Muscle Maps, re-screening provides an effective means to determine fitness improvement. Re-screening takes place either automatically as the tool prompts the user at six week intervals, as preferred by the user or personal trainer. Screen results are stored in the user's profile and are accessible for side by side comparison as indicated by FIG. 19.

The foregoing description of the invention has been presented for purposes of illustration and description. It is not intended to be exhaustive or to limit the invention to the precise form disclosed, and other modifications and variations may be possible in light of the above teachings. The embodiments were chosen and described in order to best explain the principles of the invention and its practical application to thereby enable others skilled in the art to best utilize the invention in various embodiments and various modifications as are suited to the particular use contemplated.

1. A software based, user specific, musculoskeletal fitness management tool comprising;
a means of measuring skeletal muscle strength and joint flexibility, a means of determining the overall body balance of strength and flexibility as well as a comparison of those measurements to established healthy population norms, a means presenting both qualitatively and quantitatively the results of the strength and flexibility measurements to the user, a means of automatically generating a fitness protocol designed to specifically address the findings of the strength and flexibility measurements, as well as the personal goals and predisposing health risk factors of the user, and a means of logging and monitoring the fitness protocol adherence and fitness progress.

2. The tool of claim 1 wherein muscle strength and joint flexibility are measured contains a series of body posture screens using images to indicate ideal posture as well as positive and negative deviations to which the user compares themselves, either visually, or digitally, with which healthy, moderately or severely compromised states isolated muscle groups and joints are determined.

3. The tool of claim 1 wherein qualitative results of muscle strength and joint flexibility are measured, generates a full body color coded skeletal muscle map indicating overall strength and flexibility balance as well as absolute results relative to peer normalized accepted healthy fitness levels.

4. The tool of claim 1 wherein quantitative results of muscle strength and joint flexibility are measured, generates a normalized fitness score based on measurement results, user age, body mass index (BMI) waist to hip ratio, as well as leisure and work activities and habits.

5. The tool of claim 1 wherein a fitness protocol is automatically generated, contains a database of exercises, instructions for those exercises, and images illustrating proper technique, specifically designed to strengthen targeted muscles, increase flexibility of targeted joint articulations, and/or develop coordination of muscle groups required to maintain healthy posture or movement patterns.

6. The tool of claim 1, wherein results of muscle strength and joint flexibility are measured and a fitness protocol is automatically generated, matches the results of fitness measured results with appropriate exercises contained in the database of claim 5 to create a fitness program complete with exercise dosages appropriate for the users health level, fitness level, and fitness goals.

7. The fitness program of claim 6, wherein exercises and dosages are prescribed to the users specific needs, is integrated into a personal computer or smartphone compatible calendar which contains a schedule of daily exercises as well as monitors exercise program adherence.

8. The quantitative and qualitative representation of the user's musculoskeletal health of claim 3 and claim 4 as well as exercise protocols of claim 6, and adherence of claim 7 may be stored as data tagged to the user in a central database.

9. The tool of claim 1 and claim 2 wherein the body posture screens generate a measurable representation of the user's musculoskeletal health of claim 3 and claim 4, automatically monitors improvement of musculoskeletal health by comparing present to past posture screen results stored as data identified in claim 8.

10. The tool of claim 1, daily state of health data including pain levels, energy levels, and motivation levels may be entered, which may be used either by a trainer, the user, or the tool itself to refine the user's fitness program of claim 6.
